(19) United States
(12) Patent Application Publication
(10) Pub. No.: US 2009/0070891 A1
(43) Pub. Date: Mar. 12, 2009

(54) METHODS FOR INCORPORATING MULTIPLE GENES IN A CROP PLANT

(75) Inventors: Terry Foley, Williamsburg, IA (US); Christopher Eichhorn, Williamsburg, IA (US); Arnold Rosielle, St. Louis, MO (US); Michael Graham, St. Louis, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(73) Assignee: Monsanto Technology LLC, St. Louis, MO (US)

(21) Appl. No.: 12/200,432

(22) Filed: Aug. 28, 2008

Related U.S. Application Data
(60) Provisional application No. 60/968,666, filed on Aug. 29, 2007.

(51) Int. Cl. A01H 1/00; A01H 5/00

(52) U.S. Cl. 800/263; 800/267; 800/266; 800/264; 800/265; 800/298

(57) ABSTRACT

The present invention provides haploid-based breeding methods for the integration of two or more genetic factors in a crop plant.

### CROSS-REFERENCE OF APPLICATION

This application claims benefit under 35 U.S.C. 119(e) of U.S. Provisional Application Ser. No. 60/968,666, filed Aug. 29, 2007, which is incorporated by reference herein in its entirety.

### FIELD OF INVENTION

The present invention is in the field of plant breeding. More specifically, the invention relates to methods for efficiently incorporating two or more genetic factors in a crop plant.

### BACKGROUND OF INVENTION

Traditional methods for integrating transgenic traits into plants involve backcross breeding strategies. However, as product concepts emerge for incorporating multiple transgenes per plant, new methods are needed to produce seed comprising multiple or “stacked” traits in a timely fashion. Two adaptations of the backcross approach are known and involve either use of a multiple transgene donor followed by backcrossing with selection for all traits and recurrent parent or pyramiding, i.e., initiating and continuing multiple single transgene projects with single transgene donors until all transgenic traits of the product concept are met. Both methods involve significant amounts of time and, potentially, large sample sizes to ensure recovery of all of the transgenes and equivalency to the recurrent parent. Simulation studies suggest that such backcross methods may require 8-9 generations to produce a 4-stack product incorporating four transgenic traits. Thus, there is a need in the art for reducing the time required to deliver a stacked transgenic trait hybrid to market as well as providing the potential for reducing the number of plots needed to generate an elite crop plant comprising two or more transgenic traits.

### SUMMARY OF INVENTION

The present disclosure relates to systems and methods for haploid-based breeding to integrate two or more genetic factors in a crop plant

In one embodiment, the invention provides a method for incorporating at least two genetic factors into at least one plant. The method comprises crossing a donor plant comprising at least two genetic factors with the at least one plant to obtain a plurality of progeny plants. The plurality of progeny plants are crossed with a haploid inducer line to produce induced progeny comprising haploid progeny. Haploid progeny are then selected from the induced progeny and screened for the presence of at least one marker for the at least one genetic factor and at least one marker for the genome of the at least one plant, wherein preferred haploid progeny can be selected based on the results of the screening.

The present invention includes a method for breeding of a crop plant, such as maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops, with genetic factors comprising at least one phenotype of interest, further defined as conferring a preferred property selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, or traits for improved consumer appeal.

### DETAILED DESCRIPTION

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 3 Edition, Garland Publishing, Inc.: New York, 1994; Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR § 1.822 is used.

An “allele” refers to an alternative sequence at a particular locus; the length of an allele can be as small as 1 nucleotide base, but is typically larger. Allelic sequence can be denoted as nucleic acid sequence or as amino acid sequence that is encoded by the nucleic acid sequence.

A “locus” is a position on a genomic sequence that is usually found by a point of reference; e.g., a short DNA sequence that is a gene, or part of a gene or intergenic region. A locus may refer to a nucleotide position at a reference point on a chromosome, such as a position from the end of the chromosome. The ordered list of loci known for a particular genome is called a genetic map. A variant of the DNA sequence at a given locus is called an allele and variation at a locus, i.e., two or more alleles, constitutes a polymorphism. The polymorphic sites of any nucleic acid sequence can be determined by comparing the nucleic acid sequences at one or more loci in two or more germplasm entries.

As used herein, a “nucleic acid sequence” comprises a contiguous region of nucleotides at a locus within the genome. A locus is a fixed position on a chromosome and may represent a single nucleotide, a few nucleotides or a large number of nucleotides in a genomic region. The ordered list of loci known for a particular genome is called a genetic map. A variant of the DNA sequence at a given locus is called a polymorphism. The polymorphic sites of any nucleic acid sequence can be determined by comparing the nucleic acid sequences at one or more loci in two or more germplasm entries.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found, or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In addition, the presence, absence, or variation in copy number of the preceding may comprise a polymorphism.

As used herein, the term “single nucleotide polymorphism,” also referred to by the abbreviation “SNP,” means a polymorphism at a single site wherein said polymorphism constitutes a single base pair change, an insertion of one or more base pairs, or a deletion of one or more base pairs.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics. As used herein, “genetic marker” means polymorphic nucleic acid sequence or nucleic acid feature.

As used herein, “marker assay” means a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome. As used herein, “percent recurrent parent” means percentage similarity of one or more progeny with respect to the recurrent parent. Similarity can be construed by measurement of one or more markers.

As used herein, “percent similarity” means percentage similarity of between at least one plant from one population and at least one plant from a second population based on one or more markers.

As used herein, a plant referred to as “haploid” has a single set (genome) of chromosomes and the reduced number of chromosomes (n) in the haploid plant is equal to that of the gamete.

As used herein, a plant referred to as “diploid” has two sets (genomes) of chromosomes and the chromosome number (n) is equal to that of the zygote.

As used herein, a plant referred to as “doubled haploid” is developed by doubling the haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even is the entire vegetative part of the plant does not consist of the cells with the doubled set of chromosomes; that is, a plant will be considered doubled haploid if it contains viable gametes, even if it is chimeric.

As used herein, an “inducer” is a line which when crossed with another line promotes the formation of haploid embryos. Inducers can be used male or female in a cross.

As used herein, the term “plant” includes whole plants, plant organs (i.e., leaves, stems, roots, etc.), seeds, and plant cells and progeny of the same. “Plant cell” includes without limitation seeds, suspension cultures, embryos, meristematic regions, callus tissue, leaves, shoots, gametophytes, sporophytes, pollen, and microspores.

As used herein, “phenotype” means the detectable characteristics of a cell or organism which are a manifestation of gene expression.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, the term “transgene” means nucleic acid molecules in form of DNA, such as cDNA or genomic DNA, and RNA, such as mRNA or microRNA, which may be single or double stranded.

As used herein, the term “genetic factor” can refer to a nucleic acid of interest, genetic marker, a gene, a portion of a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, a methylation pattern, and the presence, absence, or variation in copy number of any of the preceding.

As used herein, the term “inbred” means a line that has been bred for genetic homogeneity. Without limitation, examples of breeding methods to derive inbreds include pedigree breeding, recurrent selection, single-seed descent, backcrossing, and doubled haploids.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

As used herein, the term “tester” means a line used in a testcross with another line wherein the tester and the lines tested are from different germplasm pools. A tester may be isogenic or nonisogenic.

As used herein, the term “corn” means Zea mays or maize and includes all plant varieties that can be bred with corn, including wild maize species. More specifically, corn plants from the species Zea mays and the subspecies Zea mays L. ssp. Mays can be genotyped using the compositions and methods of the present invention. In an additional aspect, the corn plant is from the group Zea mays L. subsp. mays Indentata, otherwise known as dent corn. In another aspect, the corn plant is from the group Zea mays L. subsp. mays Indurata, otherwise known as flint corn. In another aspect, the corn plant is from the group Zea mays L. subsp. mays Saccharata, otherwise known as sweet corn. In another aspect, the corn plant is from the group Zea mays L. subsp. mays Amylacea, otherwise known as flour corn. In a further aspect, the corn plant is from the group Zea mays L. subsp. mays Everta, otherwise known as pop corn. Zea or corn plants that can be genotyped with the compositions and methods described herein include hybrids, inbreds, partial inbreds, or members of defined or undefined populations.

As used herein, the term “plants and parts thereof” comprise a plant, a leaf, vascular tissue, flower, pod, root, stem, seed, or a portion thereof.

As used herein, the term “comprising” means “including but not limited to”.

As used herein, an “elite line” is any line that has resulted from breeding and selection for superior agronomic performance. An elite plant is any plant from an elite line.

The present invention provides methods for delivering transgenic crop plants comprising two or more genetic factors using haploid breeding approaches. The goal of transgenic trait integration is to deliver one or more transgenic traits to an elite inbred and the typical backcross process involved multiple generations with selection at each generation for the one or more transgenic traits coupled with selection for the elite inbred, referred to as the recurrent parent. As product concepts move to transgenic trait stacks, comprising two or more transgenic traits, the trait integration process becomes exponentially more complicated because an increasing number of progeny must be screened in order to recover progeny with both the transgenic traits and, as relevant, desired percent of the recurrent parent genome (i.e., 95% recurrent parent) and minimized percent of the donor parent genome (i.e., reduce linkage drag). The methods included herein provide an advantage over the art by reducing the time required to deliver a stacked transgenic trait hybrid to market as well as providing the potential for reducing the number of plots needed to generate an elite crop plant comprising two or more transgenic traits. These methods can be applied at any point in a breeding program, wherein the “recurrent” parent can be segregating. In other aspects, the recurrent parent comprises one or more genetic factors. Further, depending on the degree of segregating in the starting material, sister line generation can occur in parallel to trait integration.

### Doubled Haploids

Plant breeding is greatly facilitated by the use of doubled haploid (DH) plants. The production of DH plants enables plant breeders to obtain inbred lines without multigenerational inbreeding, thus decreasing the time required to produce homozygous plants. A great deal of time is spared as homozygous lines are essentially instantly generated, negating the need for multigenerational conventional inbreeding.

In particular, because DH plants are entirely homozygous, they are very amenable to quantitative genetics studies. Both additive variance and additive×additive genetic variances can be estimated from DH populations. Other applications include identification of epistasis and linkage effects. Moreover, there is value in testing and evaluating homozygous lines for plant breeding programs. All of the genetic variance is among progeny in a breeding cross, which improves selection gain.

Traditional methods of producing DH plants require a high input of resources. DH plants rarely occur naturally; therefore, artificial means of production are used. First, one or more lines are crossed with an inducer parent to produce haploid seed. A number of inducer lines for maize are known in the art and include, for example, Stock 6, RWS, KEMS, KMS and ZMS, and indeterminate gametophyte (ig) mutation. In other aspects, haploid material is generated via other methods known in the art, including application of apomictic agents or other chemicals, anther culture, microspore culture, etc.

Selection of haploid seed can be accomplished by various screening methods based on phenotypic or genotypic characteristics. In one approach, material is screened with visible marker genes that are only induced in the endosperm cells of haploid cells, thus allowing for the visual identification and separation of haploid and diploid seed. Examples of visible marker genes include GFP, GUS, anthocyanin genes such as R-nj, luciferase, YFP, CFP, or CRC. Other screening approaches include chromosome counting, flow cytometry, genetic marker evaluation to infer copy number, and the like.

The resulting haploid seed, which has a haploid embryo and a normal triploid endosperm, must then undergo doubling. There are several approaches known in the art to achieve chromosome doubling. Haploid cells, haploid embryos, haploid seeds, haploid seedlings, or haploid plants can be chemically treated with a doubling agent. Non-limiting examples of known doubling agents include nitrous oxide gas, anti-microtubule herbicides, anti-microtubule agents, colchicine, pronamide, and mitotic inhibitors.

### Marker Technology

The development of markers and the association of markers with phenotypes, or quantitative trait loci (QTL) mapping for marker-assisted breeding has advanced in recent years. Examples of genetic markers are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties (U.S. Pat. No. 6,207,367). These markers form the basis for determining associations with phenotype and can be used to drive genetic gain. The implementation of marker-assisted selection is dependent on the ability to detect underlying genetic differences between individuals.

Genetic markers of the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, QTL, alleles, or genomic regions (haplotypes) that comprise or are linked to a genetic marker.

Herein, nucleic acid analysis methods are known in the art and include, but are not limited to, PCR-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form

Polymorphisms in DNA sequences can be detected or typed by a variety of effective methods well known in the art including, but not limited to, those disclosed in U.S. Pat. No. 5,468,613 and U.S. Pat. No. 5,217,863; U.S. Pat. No. 5,210,015; U.S. Pat. No. 5,876,930; U.S. Pat. No. 6,030,787; U.S. Pat. No. 6,004,744; U.S. Pat. No. 6,013,431; U.S. Pat. No. 5,595,890; U.S. Pat. No. 5,762,876; U.S. Pat. No. 5,945,283; U.S. Pat. No. 5,468,613; U.S. Pat. No. 6,090,558; U.S. Pat. No. 5,800,944; and U.S. Pat. No. 5,616,464, all of which are incorporated herein by reference in their entireties. However, the compositions and methods of this invention can be used in conjunction with any polymorphism typing method to type polymorphisms in corn genomic DNA samples. These corn genomic DNA samples used include but are not limited to corn genomic DNA isolated directly from a corn plant, cloned corn genomic DNA, or amplified corn genomic DNA.

For instance, polymorphisms in DNA sequences can be detected by hybridization to allele-specific oligonucleotide (ASO) probes as disclosed in U.S. Pat. No. 5,468,613 and U.S. Pat. No. 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. No. 6,799,122; U.S. Pat. No. 6,913,879; and U.S. Pat. No. 6,996,476.

Target nucleic acid sequence can also be detected by probe linking methods as disclosed in U.S. Pat. No. 5,616,464 employing at least one pair of probes having sequences homologous to adjacent portions of the target nucleic acid sequence and having side chains which non-covalently bind to form a stem upon base pairing of said probes to said target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. No. 6,004,744; U.S. Pat. No. 6,013,431; U.S. Pat. No. 5,595,890; U.S. Pat. No. 5,762,876; and U.S. Pat. No. 5,945,283. SBE methods are based on extension of a nucleotide primer that is immediately adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of corn genomic DNA which flanks a region containing the polymorphism to be assayed. Following amplification of the region of the corn genome containing the polymorphism, the PCR product is mixed with the third oligonucleotide (called an extension primer) which is designed to hybridize to the amplified DNA immediately adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In a preferred method for detecting polymorphisms, SNPs and Indels can be detected by methods disclosed in U.S. Pat. No. 5,210,015; U.S. Pat. No. 5,876,930; and U.S. Pat. No. 6,030,787 in which an oligonucleotide probe having a 5′fluorescent reporter dye and a 3′quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

### Marker-Assisted Breeding

Breeding has advanced from selection for economically important traits in plants and animals based on phenotypic records of an individual and its relatives to the application of molecular genetics to identify genomic regions that contain valuable genetic traits. Inclusion of genetic markers in breeding programs has accelerated the genetic accumulation of valuable traits into a germplasm compared to that achieved based on phenotypic data only. Herein, “germplasm” includes breeding germplasm, breeding populations, collection of elite inbred lines, populations of random mating individuals, and biparental crosses. Genetic marker alleles (an “allele” is an alternative sequence at a locus) are used to identify plants that contain a desired genotype at multiple loci, and that are expected to transfer the desired genotype, along with a desired phenotype to their progeny. Genetic marker alleles can be used to identify plants that contain the desired genotype at one marker locus, several loci, or a haplotype, and that would be expected to transfer the desired genotype, along with a desired phenotype to their progeny. This process has been widely referenced and has served to greatly economize plant breeding by accelerating the fixation of advantageous alleles and also eliminating the need for phenotyping every generation.

Molecular breeding is often referred to as marker-assisted selection (MAS) and marker-assisted breeding (MAB), wherein MAS refers to making breeding decisions on the basis of molecular marker genotypes and MAB is a general term representing the use of molecular markers in plant breeding. In these types of molecular breeding programs, genetic marker alleles can be used to identify plants that contain the desired genotype at one marker locus, several loci, or a haplotype, and that would be expected to transfer the desired genotype, along with a desired phenotype to their progeny. Markers are highly useful in plant breeding because once established, they are not subject to environmental or epistatic interactions. Furthermore, certain types of markers are suited for high throughput detection, enabling rapid identification in a cost effective manner.

Marker discovery and development in crops provides the initial framework for applications to MAB (U.S. Pat. No. 5,437,697; US Patent Application 2005/0204780, US Patent Application 2005/0216545, US Patent Application 2005/0218305). The resulting “genetic map” is the representation of the relative position of characterized loci (DNA markers or any other locus for which alleles can be identified) along the chromosomes. The measure of distance on this map is relative to the frequency of crossover events between sister chromatids at meiosis. As a set, polyallelic markers have served as a useful tool for fingerprinting plants to inform the degree of identity of lines or varieties (U.S. Pat. No. 6,207,367). These markers form the basis for determining associations with phenotype and can be used to drive genetic gain. The implementation of MAS, wherein selection decisions are based on marker genotypes, is dependent on the ability to detect underlying genetic differences between individuals.

Many individuals and companies have developed versions of molecular breeding. One common aspect is that molecular breeding relies on markers to report differences which are then used to make selections. However, these markers provide no or very limited information on the differences at the DNA sequence level; for example, a typical biallelic SNP marker provides information on only one base pair position and it can only distinguish between 2, rather than 4, nucleotides. Using expression profile assays gives the power to query 4 nucleotides at any given position within a nucleic acid sequence as directed by inclusion of target nucleic acid sequences. Furthermore, this power will be useful to fingerprint plant populations or lineages to allow genome wide discovery of useful variation, build pedigrees or calculate breeding values.

Further, the present invention contemplates that preferred plants comprising at least one genotype of interest are identified for advancement in transgenic trait integration using the methods disclosed in PCT/US07/18101 (filed Aug. 15, 2007) claiming priority to U.S. Provisional Application Ser. No. 60/837,864 (filed Aug. 15, 2006), both of which are incorporated herein by reference in their entirety, wherein a genotype of interest may correspond to a QTL or haplotype and is associated with at least one phenotype of interest. In other aspects, preferred transgenic events are selected based on linkage with one or more preferred haplotypes based on predicted performance for at least one phenotypic trait, i.e., yield, as disclosed in U.S. Patent Application US2006/0282911, which is incorporated herein by reference in its entirety. In another aspect, the genotype of interest corresponds to a transgene modulating locus, as disclosed in co-owned U.S. patent application Ser. No. 12/144,278, filed Jun. 23, 2008, which is incorporated herein by reference in its entirety.

The methods include association of at least one haplotype with at least one phenotype, wherein the association is represented by a numerical value and the numerical value is used in the decision-making of a breeding program. Non-limiting examples of numerical values include haplotype effect estimates, haplotype frequencies, and breeding values. In the present invention, it is particularly useful to identify haploid plants of interest based on at least one genotype, such that only those lines undergo doubling, which saves resources. Resulting doubled haploid plants comprising at least one genotype of interest are then advanced in a breeding program for use in activities related to germplasm improvement. In another aspect, it is particularly useful to implement these methods to identify recipient lines of interest, i.e., the recurrent parent.

Genotyping can be further economized by high throughput, non-destructive seed sampling. In one embodiment, plants can be screened for one or more markers, such as genetic markers, using high throughput, non-destructive seed sampling. In a preferred aspect, haploid seed is sampled in this manner and only seed with at least one marker genotype of interest is advanced for doubling. Apparatus and methods for the high throughput, non-destructive sampling of seeds have been described which would overcome the obstacles of statistical samples by allowing for individual seed analysis. For example, commonly-owned U.S. patent application Ser. No. 11/213,430 (filed Aug. 26, 2005); U.S. patent application Ser. No. 11/213,431 (filed Aug. 26, 2005); U.S. patent application Ser. No. 11/213,432 (filed Aug. 26, 2005); U.S. patent application Ser. No. 11/213,434 (filed Aug. 26, 2005); U.S. patent application Ser. No. 11/213,435 (filed Aug. 26, 2005), U.S. patent application Ser. No. 11/680,611 (filed Mar. 2, 2007), and U.S. patent application Ser. No. 12/128,279 (filed May 28, 2008), which are incorporated herein by reference in their entirety, disclose apparatus and systems for the automated sampling of seeds as well as methods of sampling, testing and bulking seeds.

In a preferred embodiment of the present invention, high throughput, non-destructive seed sampling, for example, as described in commonly-owned U.S. patent application Ser. No. 11/680,611 and U.S. patent application Ser. No. 12/128,279, is used for sampling plants of the present invention. This sampling platform permits the rapid identification of seed comprising preferred genotypes or phenotypic characters such that only preferred or targeted seed is planted, saving resources on greenhouse and/or field plots. In particular, when haploid seed is sampled using high throughput, non-destructive seed sampling, resources are saved by only advancing preferred seed for doubling, such as seed comprising the transgenic traits of the donor and desired percent of the recurrent parent genome.

### Plant Breeding

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). A cultivar is a race or variety of a plant species that has been created or selected intentionally and maintained through cultivation.

The present invention provides for parts of the plants of the present invention.

Selected, non-limiting approaches for breeding the plants of the present invention are set forth below. A breeding program can be enhanced using marker assisted selection (MAS) on the progeny of any cross. It is understood that nucleic acid markers of the present invention can be used in a MAS (breeding) program. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

In one aspect, MAB programs use a plurality of markers to identify higher performing selections that have, on average, a higher frequency of favorable alleles at one or more loci. Fingerprinting was developed to determine the genome-wide marker distribution. Using the resulting marker distance and/or marker similarities indices between two or more lines, it is possible to build pedigrees and to calculate the breeding value across all assessed loci. Herein, breeding values are calculated based on expression profile effect estimates and expression profile (i.e., allele) frequency, wherein the expression profile breeding value represents the effect of fixing a particular nucleic acid sequence (i.e., allele) underlying the expression profile in a population, thus providing the basis for ranking nucleic acid sequences, based on corresponding expression profiles.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

For hybrid crops, the development of new elite hybrids requires the development and selection of elite inbred lines, the crossing of these lines and selection of superior hybrid crosses. The hybrid seed can be produced by manual crosses between selected male-fertile parents or by using male sterility systems. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

The doubled haploid (DH) approach achieves isogenic plants in a shorter time frame. DH plants provide an invaluable tool to plant breeders, particularly for generating inbred lines and quantitative genetics studies. For breeders, DH populations have been particularly useful in QTL mapping, cytoplasmic conversions, and trait introgression. Moreover, there is value in testing and evaluating homozygous lines for plant breeding programs. All of the genetic variance is among progeny in a breeding cross, which improves selection gain.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Manograph., 16:249, 1987; Fehr, “Principles of variety development,” Theory and Technique, (Vol. 1) and Crop Species Soybean (Vol. 2), Iowa State Univ., Macmillan Pub. Co., NY, 360-376, 1987).

### Transgenic Breeding

### 1. Methods and Compositions for Recombinant Nucleic Acids

Nucleic acids for proteins disclosed in the present invention can be expressed in plant cells by operably linking them to a promoter functional in plants Tissue specific and/or inducible promoters may be utilized for appropriate expression of a nucleic acid for a particular trait. The 3′ un-translated sequence, 3′ transcription termination region, or polyadenylation region means a DNA molecule linked to and located downstream of a structural polynucleotide molecule responsible for a transgenic trait and includes polynucleotides that provide polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. A 5′ UTR that functions as a translation leader sequence is a DNA genetic element located between the promoter sequence and the coding sequence. The translation leader sequence is present in the fully processed mRNA upstream of the translation start sequence. The translation leader sequence may affect processing of the primary transcript to mRNA, mRNA stability or translation efficiency.

The nucleic acids of proteins encoding transgenic traits are operably linked to various expression elements to create expression unit. These expression units generally comprise in 5′ to 3′ direction: a promoter, nucleic acid for a trait, a 3′ untranslated region (UTR). Several other expression elements such as a 5′UTRs, organellar transit peptide sequences, and introns may be added to facilitate expression of the trait. In some embodiments, protein product of a nucleic acid responsible for a particular transgenic trait is targeted to an organelle for proper functioning. For example, targeting of a protein to chloroplast is achieved by using a chloroplast transit peptide sequences. These sequences can be isolated or synthesized from amino acid or nucleic acid sequences of nuclear encoded by chloroplast targeted genes such as small subunit (RbcS2) of ribulose-1,5,-bisphosphate carboxylase, ferredoxin, ferredoxin oxidoreductase, the light-harvesting complex protein I and protein II, and thioredoxin F proteins. Other examples of chloroplast targeting sequences include the maize cab-m7 signal sequence (Becker, et al., 1992; PCT WO 97/41228), the pea glutathione reductase signal sequence (Creissen, et al., 1995; PCT WO 97/41228), and the CTP of the Nicotiana tobaccum ribulose 1,5-bisphosphate carboxylase small subunit chloroplast transit peptide (NtSSU-CTP) (Mazur, et al., 1985).

The term “intron” refers to a polynucleotide molecule that may be isolated or identified from the intervening sequence of a genomic copy of a gene and may be defined generally as a region spliced out during mRNA processing prior to translation. Alternately, introns may be synthetically produced. Introns may themselves contain sub-elements such as cis-elements or enhancer domains that effect the transcription of operably linked genes. A “plant intron” is a native or non-native intron that is functional in plant cells. A plant intron may be used as a regulatory element for modulating expression of an operably linked gene or genes. A polynucleotide molecule sequence in a transformation construct may comprise introns. The introns may be heterologous with respect to the transcribable polynucleotide molecule sequence. Examples of introns include the corn actin intron and the corn HSP70 intron (U.S. Pat. No. 5,859,347, herein incorporated by reference).

Duplication of any expression element across various expression units is avoided due to transgenic trait silencing or related effects. Duplicated elements across various expression units are used only when they did not interfere with each other or did not result into silencing of a transgenic trait.

Methods are known in the art for assembling and introducing constructs into a cell in such a manner that the nucleic acid molecule for a transgenic trait is transcribed into a functional mRNA molecule that is translated and expressed as a protein product. For the practice of the present invention, conventional compositions and methods for preparing and using constructs and host cells are well known to one skilled in the art, see for example, Molecular Cloning: A Laboratory Manual, 3rd edition Volumes 1, 2, and 3 (2000) J. F. Sambrook, D. W. Russell, and N. Irwin, Cold Spring Harbor Laboratory Press. Methods for making transformation constructs particularly suited to plant transformation include, without limitation, those described in U.S. Pat. No. 4,971,908, U.S. Pat. No. 4,940,835, U.S. Pat. No. 4,769,061 and U.S. Pat. No. 4,757,011, all of which are herein incorporated by reference in their entirety. These types of vectors have also been reviewed (Rodriguez, et al., Vectors: A Survey of Molecular Cloning Vectors and Their Uses, Butterworths, Boston, 1988; Glick, et al., Methods in Plant Molecular Biology and Biotechnology, CRC Press, Boca Raton, Fla., 1993).

Normally, the expression units are provided between one or more T-DNA borders on a transformation construct. The transformation constructs permit the integration of the expression unit between the T-DNA borders into the genome of a plant cell. The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, LBA4404, EHA101, and EHA105 carrying a plasmid having a transfer function for the expression unit. Other strains known to those skilled in the art of plant transformation can function in the present invention.

The transgenic traits of the present invention are introduced into inbreds by transformation methods known to those skilled in the art of plant tissue culture and transformation. Any of the techniques known in the art for introducing expression units into plants may be used in accordance with the invention. Examples of such methods include electroporation as illustrated in U.S. Pat. No. 5,384,253; microprojectile bombardment as illustrated in U.S. Pat. No. 5,015,580; U.S. Pat. No. 5,550,318; U.S. Pat. No. 5,538,880; U.S. Pat. No. 6,160,208; U.S. Pat. No. 6,399,861; and U.S. Pat. No. 6,403,865; protoplast transformation as illustrated in U.S. Pat. No. 5,508,184; and Agrobacterium-mediated transformation as illustrated in U.S. Pat. No. 5,635,055; U.S. Pat. No. 5,824,877; U.S. Pat. No. 5,591,616; U.S. Pat. No. 5,981,840; and U.S. Pat. No. 6,384,301.

After effecting delivery of expression units to recipient cells, the next steps generally concern identifying the transformed cells for further culturing and plant regeneration. In order to improve the ability to identify transformants, one may desire to employ a selectable or screenable marker gene with a transformation construct prepared in accordance with the invention. In this case, one would then generally assay the potentially transformed cell population by exposing the cells to a selective agent or agents, or one would screen the cells for the desired marker gene trait. Examples of various selectable or screenable markers are disclosed in Miki and McHugh, 2004, Selectable marker genes in transgenic plants: applications, alternatives and biosafety, Journal of Biotechnology, 107, 193.

Cells that survive the exposure to the selective agent, or cells that have been scored positive in a screening assay, may be cultured in media that supports regeneration of plants. In an exemplary embodiment, any suitable plant tissue culture media, for example, MS and N6 media may be modified by including further substances such as growth regulators. Tissue may be maintained on a basic media with growth regulators until sufficient tissue is available to begin plant regeneration efforts, or following repeated rounds of manual selection, until the morphology of the tissue is suitable for regeneration, then transferred to media conducive to shoot formation. Cultures are transferred periodically until sufficient shoot formation had occurred. Once shoots are formed, they are transferred to media conducive to root formation. Once sufficient roots are formed, plants can be transferred to soil for further growth and maturity.

To confirm the presence of the DNA for a transgenic trait in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

TABLE 1
Non-limiting examples of transgenic traits that can be used in accordance with
the methods of the present invention to identify preferred germplasm and transgene
combinations.

| Trait | Gene/protein | Reference |
|---|---|---|
| Herbicide | 5-enolpyruvylshikimate-3- | U.S. Pat. Nos. 5,627,061, |
| tolerance | phosphate synthases | 5,633,435, 6,040,497, 5,094,945, |
| | | 6,825,400; US20060143727; |
| | | WO04009761 |
| | glyphosate oxidoreductase (GOX) | U.S. Pat. No. 5,463,175 |
| | glyphosate decarboxylase | WO05003362 and U.S. Patent |
| | | Application 20040177399 |
| | glyphosate-N-acetyl transferase | U.S. Patent Applications |
| | (GAT) | 20030083480, 20060200874 |
| | dicamba monooxygenase | U.S. Patent Applications |
| | | 20030115626, 20030135879 |
| | phosphinothricin acetyltransferase | U.S. Pat. Nos. 5,646,024, |
| | (bar) | 5,561,236, 5,637,489, 5,276,268, |
| | | and 5,273,894; EP 275,957 |
| | 2,2-dichloropropionic acid | WO9927116 |
| | dehalogenase | |
| | acetohydroxyacid synthase or | U.S. Pat. Nos. 6,225,105, |
| | acetolactate synthase | 5,767,366, 4,761,373, 5,633,437, |
| | | 6,613,963, 5,013,659, 5,141,870, |
| | | 5,378,824, 5,605,011 |
| | haloarylnitrilase (Bxn) | U.S. Pat. No. 4,810,648 |
| | acetyl-coenzyme A carboxylase | U.S. Pat. No. 6,414,222 |
| | (seq IDs) | |
| | dihydropteroate synthase (sul I) | U.S. Pat. Nos. 5,597,717, |
| | | 5,633,444, 5,719,046 |
| | 32 kD photosystem II polypeptide | Hirschberg et al., 1983, Science, |
| | (psbA) | 222: 1346-1349 |
| | anthranilate synthase | U.S. Pat. No. 4,581,847 |
| | phytoene desaturase (crtI) | JP06343473 |
| | hydroxy-phenyl pyruvate | U.S. Pat. No. 6,268,549 |
| | dioxygenase | |
| | protoporphyrinogen oxidase I | U.S. Pat. No. 5,939,602 |
| | (protox) | |
| | aryloxyalkanoate dioxygenase | WO05107437 |
| | (AAD-1)(Seq IDs) | |
| Male/female | Several | U.S. Patent Application |
| sterility system | | 20050150013 |
| | Glyphosate/EPSPS | U.S. Pat. No. 6,762,344 |
| | Male sterility gene linked to | U.S. Pat. No. 6,646,186 |
| | herbicide resistant gene | |
| | Acetylated toxins/deacetylase | U.S. Pat. No. 6,384,304 |
| | Antisense to an essential gene in | U.S. Pat. No. 6,255,564 |
| | pollen formation | |
| | DNAase or endonuclease/restorer | U.S. Pat. No. 6,046,382 |
| | protein | |
| | Ribonuclease/barnase | U.S. Pat. No. 5,633,441 |
| Intrinsic yield | glycolate oxidase or glycolate | U.S. Patent Application |
| | dehydrogenase, glyoxylate | 2006009598 |
| | carboligase, tartronic | |
| | semialdehyde reductase | |
| | eukaryotic initiation Factor 5A; | U.S. Patent Application |
| | deoxyhypusine synthase | 20050235378 |
| | zinc finger protein | U.S. Patent Application |
| | | 20060048239 |
| | methionine aminopeptidase | U.S. Patent Application |
| | | 20060037106 |
| | Several | U.S. Patent Application |
| | | 20060037106 |
| | 2,4-D dioxygenase | U.S. Patent Application |
| | | 20060030488 |
| | Serine carboxypeptidase | U.S. Patent Application |
| | | 20060085872 |
| | Several | U.S. RE38,446; U.S. Pat. Nos. |
| | | 6,716,474, 6,663,906, 6,476,295, |
| | | 6,441,277, 6,423,828, 6,399,330, |
| | | 6,372,211, 6,235,971, 6,222,098, |
| | | 5,716,837, 6,723,897, 6,518,488 |
| Nitrogen use | fungal nitrate reductases, mutant | U.S. Patent Application |
| efficiency | nitrate reductases lacking post- | 20050044585 |
| | translational regulation, glutamate | |
| | synthetase-1, | |
| | glutamate dehydrogenase, | |
| | aminotransferases, nitrate | |
| | transporters (high affinity and low | |
| | affinities), ammonia transporters | |
| | and amino acid transporters | |
| | glutamate dehydrogenase | U.S. Patent Application |
| | | 20060090219 |
| | cytosolic glutamine synthetase; | EP0722494 |
| | root-specific glutamine | |
| | synthetase. | |
| | Several | WO05103270; U.S. Patent |
| | | Applications 20070044172, |
| | | 20070107084 |
| | glutamate 2-oxoglutarate | U.S. Pat. No. 6,864,405 |
| | aminotransferase | |
| Abiotic Stress | succinate semialdehyde | U.S. Patent Application |
| tolerance | dehydrogenase | 20060075522 |
| including cold, | several | U.S. Pat. Nos. S,792,921, |
| heat, drought | | 6,051,755, 7,084,323, 6,229,069, |
| | | 6,534,446, 6,951,971, 6,376,747, |
| | | 6,624,139, 6,559,099, 6,455,468, |
| | | 6,635,803, 6,515,202, 6,960,709, |
| | | 6,706,866, 7,164,057, 7,141,720, |
| | | 6,756,526, 6,677,504, 6,689,939, |
| | | 6,710,229, 6,720,477, 6,818,805, |
| | | 6,867,351, 7,074,985, 7,091,402, |
| | | 7,101,828, 7,138,277, 7,154,025, |
| | | 7,161,063, 7,166,767, 7,176,027, |
| | | 7,179,962, 7,186,561, 7,186,563, |
| | | 7,186,887, 7,193,130; U.S. Patent |
| | | Applications 2003/0221224, |
| | | 2004/0128712, 2004/0187175, |
| | | 2005/0097640, 2005/0204431, |
| | | 2005/0235382, 2005/0246795, |
| | | 2005/0086718, 20060008874, |
| | | 2006/0015972, 2006/0021082, |
| | | 2006/0021091, 2006/0026716, |
| | | 2006/0064775, 2006/0064784, |
| | | 2006/0075523, 2006/0112454, |
| | | 2006/0123516, 2006/0137043, |
| | | 2006/0150285, 2006/0168692, |
| | | 2006/0162027, 2006/0183137, |
| | | 2006/0183137, 2006/0185038, |
| | | 2006/0253938, 2007/0006344, |
| | | 2007/0006348, 2007/0079400, |
| | | 2007/0028333, 20070107084; |
| | | WO06032708 |
| | transcription factor | U.S. Patent Application |
| | | 20060162027 |
| Disease | CYP93C (cytochrome P450) | U.S. Pat. No. 7,038,113 |
| resistance | Several | U.S. Pat. Nos. 7,038,113; |
| | | 6,653,280; 6,573,361; 6,506,962; |
| | | 6,316,407; 6,215,048; 5,516,671; |
| | | 5,773,696; 6,121,436; 6,316,407; |
| | | 6,506,962; 6,617,496; 6,608,241; |
| | | 6,015,940; 6,013,864; 5,850,023; |
| | | 5,304,730; 6,228,992; 5,516,671 |
| Insect resistance | Several | U.S. Pat. Nos. 7,049,491, |
| | | 6,809,078, 6,713,259, 6,713,063, |
| | | 6,686,452, 6,657,046, 6,645,497, |
| | | 6,642,030, 6,639,054, 6,620,988, |
| | | 6,593,293, 6,555,655, 6,538,109, |
| | | 6,537,756, 6,521,442, 6,501,009, |
| | | 6,468,523, 6,326,351, 6,313,378, |
| | | 6,284,949, 6,281,016, 6,248,536, |
| | | 6,242,241, 6,221,649, 6,177,615, |
| | | 6,156,573, 6,153,814, 6,110,464, |
| | | 6,093,695, 6,063,756, 6,063,597, |
| | | 6,023,013, 6,002,068, 5,959,091, |
| | | 5,942,664, 5,942,658, 5,880,275, |
| | | 5,763,245, 5,763,241; |
| | | WO05059103; U.S. Patent |
| | | Applications 20060037095, |
| | | 20060095986, 20050039226, |
| | | 20060070139, 20060021087, |
| | | 60808834 |
| Enhanced amino | glutamate dehydrogenase | U.S. Pat. No. 6,969,782 |
| acid content | threonine deaminase | U.S. Patent Application |
| | | 20050289668 |
| | dihydrodipicolinic acid synthase | U.S. Pat. Nos. 5,258,300, |
| | (dap A) | 6,329,574, 7,157,281 |
| | chymotrypsin inhibitor | U.S. Pat. No. 6,800,726 |
| Enhanced | Several | U.S. Patent Application |
| protein | | 20050055746 |
| content | | |
| Modified fatty | Several | U.S. Pat. Nos. 6,444,876, |
| acids | | 6,426,447, 6,380,462, 6,949,698, |
| | | 6949698, 6,828,475, 6,822,141, |
| | | 6,770,465, 6,706,950, 6,660,849, |
| | | 6,596,538, 6,589,767; 6,537,750, |
| | | 6,489,461, 6,459,018 |
| Enhanced oil | Several | U.S. Pat. Nos. 6,495,739, |
| content | | 5,608,149, 6,483,008, 6,476,295, |
| | | 6,822,141, 7,135,617 |
| Carbohydrate | raffinose saccharides | U.S. Pat. No. 6,967,262 |
| production | | |
| Starch | Several | U.S. Pat. No. 6,951,969; |
| Production | | 6,538,181; 6,538,179; 6,538,178; |
| | | 5,750,876; 6,476,295 |
| Phytic acid | inositol polyphosphate 2-kinase | WO06029296 |
| reduction | inositol 1,3,4-triphosphate 5/6- | U.S. Patent Application |
| | kinases | 20050202486 |
| Processing | Several | WO05096804; U.S. Pat. No. |
| enzymes | | 5,543,576 |
| production | | |
| Biopolymers | Several | U.S. RE37,543; U.S. Pat. Nos. |
| | | 6,228,623, 5,958,745 and U.S. |
| | | Patent Application 20030028917 |
| Enhanced | Several | U.S. Pat. Nos. 6,723,837, |
| Nutrition | | 6,653,530, 6,5412,59, 5,985,605, |
| | | 6,171,640 |
| Pharmaceutical | Several | U.S. Pat. Nos. 6,812,379, |
| peptides and | | 6,774,283, 6,140,075, 6,080,560 |
| secretable | | |
| peptides | | |
| Improved | sucrose phosphorylase | U.S. Pat. No. 6,476,295 |
| processing trait | | |
| Improved | thioredoxin and/or thioredoxin | U.S. Pat. No. 6,531,648 |
| digestibility | reductase | |

### 2. Transgenic Trait Integration

Once a transgene for a trait has been introduced into a plant, that gene can be introduced into any plant sexually compatible with the first plant by crossing, without the need for directly transforming the second plant. Therefore, as used herein the term “progeny” denotes the offspring of any generation of a parent plant prepared in accordance with the present invention. A “transgenic plant” may thus be of any generation.

As cited above, descriptions of breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Manograph., 16:249, 1987; Fehr, “Principles of variety development,” Theory and Technique, (Vol 1) and Crop Species Soybean (Vol 2), Iowa State Univ., Macmillian Pub. Co., NY, 360-376, 1987).

In general, two distinct breeding stages are used for commercial development of elite cultivars containing a transgenic trait. The first stage involves evaluating and selecting a superior transgenic event, while the second stage involves integrating the selected transgenic event in a commercial germplasm.

In a typical transgenic breeding program, a transformation construct responsible for a transgenic trait is introduced into the genome via a transformation method. Numerous independent transformants (events) are usually generated for each construct. These events are evaluated to select those with superior performance. The event evaluation process is based on several criteria including 1) transgene expression/efficacy of the transgenic trait, 2) molecular characterization of the trait, 3) segregation of the trait, 4) agronomics of the developed event, and 5) stability of the transgenic trait expression. Evaluation of large populations of independent events and more thorough evaluation result in the greater chance of success.

Events showing right level of protein expression that corresponds with right phenotype (efficacy) are selected for further use by evaluating the event for insertion site, transgene copy number, intactness of the transgene, zygosity of the transgene, level of inbreeding associated with a genotype, and environmental conditions. Events showing a clean single intact insert are found by conducting molecular assays for copy number, insert number, insert complexity, presence of the vector backbone, and development of event-specific assays and are used for further development. Segregation of the trait is tested to select transgenic events that follow a single-locus segregation pattern. Segregation can be evaluated directly by assessing the segregation of the transgenic trait or indirectly by assessing segregation of a selectable marker (associated with the transgenic trait).

Event instability over generations is often caused by transgene inactivation due to multiple transgene copies, zygosity level, highly methylated insertion sites, or level of stress. Thus, stability of transgenic trait expression is ascertained by testing in different generations, environments, and in different genetic backgrounds. Events that show transgenic trait silencing are discarded.

Generally, events with a single intact insert that inherited as a single dominant gene and follow Mendelian segregation ratios are used in commercial transgenic trait integration strategies such as backcrossing and forward breeding.

In another aspect, testing may be expanded to assess at least one lead event in at least two different genetic backgrounds in at least two different locations for the purpose of evaluation of genotype interactions with the one or more transgenes in two or more locations.

In another aspect, testing may be expanded to assess at least one lead event in at least two different genetic backgrounds in at least two different conditions for at least one environmental factor for the purpose of evaluation of genotype interactions with the one or more transgenes in two or more environmental conditions.

In one embodiment, transgenic trait integration is accomplished using backcrossing to recover the genotype of an elite inbred with an additional transgenic trait. In each backcross generation, plants that contain the transgene are identified and crossed to the elite recurrent parent. Several backcross generations with selection for recurrent parent phenotype are generally used by commercial breeders to recover the genotype of the elite parent with the additional transgenic trait. During backcrossing the transgene is kept in a hemizygous state. Therefore, at the end of the backcrossing, the plants are self- or sib-pollinated to fix the transgene in a homozygous state. The number of backcross generations can be reduced by molecular assisted backcrossing (MABC). The MABC method uses genetic markers to identify plants that are most similar to the recurrent parent in each backcross generation. With the use of MABC and appropriate population size, it is possible to identify plants that have recovered over 98% of the recurrent parent genome after only two or three backcross generations. By eliminating several generations of backcrossing, it is often possible to bring a commercial transgenic product to market one year earlier than a product produced by conventional backcrossing.

In a preferred embodiment, MABC also targets markers corresponding at least one transgene modulating locus, previously identified from marker-trait mapping in a panel of germplasm entries segregating for transgene modulators. In another embodiment, MAS is used in activities related to line development in order to develop elite lines with preferred transgene modulating genotypes. In another aspect, additional markers may be used in selection decisions that are associated with the transgene modulating loci and can be detected by means of visual assays, chemical or analytic assays, or some other type of phenotypic assay.

Forward breeding is any breeding method that has the goal of developing a transgenic variety, inbred line, or hybrid that is genotypically different, and superior, to the parents used to develop the improved genotype. When forward breeding a transgenic crop, selection pressure for the efficacy of the transgene is usually applied during each generation of the breeding program.

In a preferred aspect, inbred lines used in the present invention for transgenic trait integration are prepared using the stacking strategy methods disclosed in the U.S. Provisional Application Ser. Nos. 60/848,952 and 60/922,013 (filed Oct. 3, 2006 and Apr. 5, 2007 respectively), which are incorporated herein by referenced in their entirety, to produce transgenic inbred parents in order to develop hybrid product concepts with preferred economic value.

### EXAMPLES

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

### Example 1

### Stacking at Least Two Genetic Factors Using a Haploid Approach

There is tremendous value in the hybrid corn market for products with at least two transgenic traits, such as herbicide tolerance and insect resistance. However, traditional methods relying solely on backcross breeding will result in an exponential increase in resources needed to deliver hybrids with two or more genetic factors, in terms of years to market, plots needed, etc. In the present example, the methods of this invention are detailed, wherein an expedited approach for breeding and transgenic trait integration involving the use of the DH process are provided. The present invention provides a combination of breeding methods directed to recovery of the at least two genetic factors of interest with maximized recovery of recurrent parent of at least 95%, and in preferred aspects, at least 98%.

In one embodiment, a new line, for example “Line A”, can be developed and readied for transgenic trait integration to begin marker assisted backcrossing. The donor line contains at least 2 transgenic traits which are unlinked to one another; notably, in other aspects, 4 or more transgenic traits are targeted and in another aspect, two or more transgenic traits are genetically linked. In one aspect, the donor and new line are related to one another and the coefficient of similarity is 80%. In another aspect, similarity between donor and new line are greater than 50% and less than 100%. In some aspects, the similarity between any donor and any new line is 60%, 70%, 80%, 90%, 91%, 92%, 93%, 94%, 95%, 96%, 97%, 98%, 99%, or 99.5%.

In other aspects, Line A may not be fully inbred and is segregating at one or more loci. Thus, the F1 progeny are screened not only for the presence of the genetic factors of interest but can also be evaluated for breeding decisions in terms of line development and subsequent sister line generation.

The present invention contemplates that two or more donors are available with different numbers and types of genetic factors, as well as different genetic backgrounds, can be created to facilitate the process and transfer of different gene combinations in order to avoid null transgene issues and to facilitate varied product concepts. Further, donor sets can be created for specific maturity groups as well as similarity (i.e., a donor set for genetic clusters in a germplasm pool). In certain aspects, the donor is the transformation line and in other aspects the donor is a conversion.

In another aspect, a set of donor s are developed to correspond to the genetic diversity of the germplasm pool, such that conversions can be initiated with donors and recurrent parents that are at least 75% similar. In other aspects, two lines are at least 85% similar. In other aspects, the two lines are at least 95% similar. Greater similarity provides the advantage of fewer MABC cycles to recover recurrent parent.

The present examples provides phases of a stacked trait integration program, wherein the inventors contemplate that at the second phase and beyond, one or more backcross generations may be introduced for the purpose of maximizing recovery of recurrent parent, herein referred to as “Line A” for the purpose of illustration.

In one embodiment, the F1 is made by crossing a donor with four transgenic traits by “Line A” in the first phase, wherein the donor and Line A are 80% identical. For purposes of illustration 500 kernels of this cross are produced. Next, at the second phase, the F1 can undergo at least one generation of backcrossing to the recurrent parent, followed by selection of progeny with maximum percent recurrent parent. In one aspect of the present invention, in the second phase, the F1 is planted in a maternal induction crossing situation using the F1 as female and a haploid inducer line as male. In another aspect, the F1 is used as female and crossed with a male haploid inducer line in a paternal induction cross. This invention anticipates haploid plants can be generated using various methods known in the art. For the purposes of illustration, if 500 kernels from above are planted, one can conservatively estimate that 75,000 seeds would be produced (500 plants×150 seeds per ear=75,000 seeds) and, of these, approximately 3,500 to 4,000 would be putative haploids (75,000 induced seeds×0.05 induction=3,750 putative haploids).

Putative haploid kernels are identified using visual screening, phenotypic screening, and/or genotypic screening using methods known in the art. In a preferred aspect of the present invention, each of the putative haploid kernels is sampled using high throughput, non-destructive seed sampling to determine that each of the transgenic traits of the donor is present and that recurrent parent (RP) is maximized before planting in order to economize plots. Theoretically, 1 of 16 of the putative haploid kernels produced will contain all four transgenic traits (4,000 putative haploids/16=250 putative haploid kernels that have all 4 traits) and, on average, half of these will be higher than 90% RP (125 putative haploid kernels). For example, in this example, it would be cost efficient to screen for the four traits first on the 4,000 putative haploid kernels to narrow the field of focus before examining recurrent parent on the remaining 250 putative haploid kernels that contain all four traits.

Notably, haploid kernels are an ideal material for transgenic trait integration since regions are homozygous and the hemizygous condition that is commonly dealt with in backcrossing programs is eliminated. This provides great advantages in backcrossing approaches. It is possible to accurately identify which regions are fixed and which regions need to be changed in the next cross. It is possible marker optimization could take place after this step to reduce conversion cost.

In this case, since RP and donor are 80% identical by descent, and the goal in the second phase is to advance only those kernels that contained all four traits and are greater than 95% RP, it may be preferred to increase the number of haploid kernels produced. Therefore, in one aspect, the second phase presents an opportunity to induce more plants to increase the probability of the desired progeny (i.e., all transgenic traits and preferred percent RP). For instance, it could be possible that induction of 1000 plants instead of 500 may actually lead to enough kernels that contain all four transgenic traits and are above 98% RP. These resultant kernels can be advanced to a doubling nursery.

In the case of the four trait model, this could eliminate the need for subsequent generations. In another aspect, if the donor(s) used are more similar to RP to begin with, numbers of haploid kernels required can be reduced. As the number of transgenic traits increases above 4 to say, 8, the amount of haploids necessary to do this in a single step increases.

The current example illustrates a stepwise progression that increases percentage of recurrent parent while being less costly and more amenable to incorporation of higher numbers of genetic factors. This may become necessary as the number of transgenic traits involved is increased.

In the third phase, selected putative haploid kernels are planted in a nursery next to “Line A,” wherein 100 or more putative haploids are used. Non-limiting examples of the subsequent steps are below:

Option 1: In one aspect, it may be advantageous to select only the putative haploid kernels that contain all 4 traits and the highest amount of recurrent parent, wherein these individuals are doubled and then crossed to Line A. Since marker selection has been employed, one generation can be skipped in the pre-commercial pipeline by leveraging genotyping and high throughput, non-destructive seed sampling. At this point, the selected putative haploid kernels undergo the doubling process. Haploid kernels undergo doubling using methods known in the art.

At the same time, “Line A” is planted in a nursery leaving space in an adjacent row for the transplants of the potted seedlings. Timing of planting of “Line A” will most likely occur when the haploid seedlings are fairly well recovered, accounting for the stress of the “doubling” process. As such, it may be necessary to delay planting of “Line A” in order to ensure proper nick; for example, it may be necessary to plant “Line A” slightly ahead of the transplant date.

At the time of pollination, the putative doubled haploid seedlings will produce limited amounts of pollen and will be used as a male donor onto “Line A”. The cross is made only in this direction. Based on historic survival rates, one skilled in the art would believe that a subset of transplants will survive to the field, and, of that subset, generally more than half shed pollen. It is possible to generate enough kernels (i.e., at least 500 kernels) in this method to advance to phase four using only 2-13K rows. It is also possible that the haploid kernels that shed can be selfed in the case of individuals that are exceptionally high in recurrent parent.

In the event that the nick is off, risk can be reduced by making the cross in the manner described in Option 2 below; with the only difference being that the haploid plants will shed limited amounts of pollen.

Option 2: In a second embodiment, the present invention contemplates that the haploid progeny from phase two will be directly backcrossed onto Line A. In this scenario, the selected haploid kernels are used as female and crossed by “Line A.” Haploid plants, generally have low amounts of male fertility, but readily produce silk. The plants will set seed, but in limited quantities. For example, in the case 90 of the 125 plants are pollinated, ⅓ of these pollinations would produce seed (90 pollinations×0.33=30 ears), and, on average, 10 seeds per ear (30 ears×10 seeds=300 seeds). Each of these seeds contains all four transgenic traits and is, at a minimum, 95% recurrent parent. While this approach does not produce as much seed as Option 1, it does have the advantage of requiring less management and minimizes the risk of miss-nick.

Option 3: In another embodiment, the putative haploid seed is doubled and reciprocal crosses with Line A are made. Doubled haploid plants will produce limited amounts of pollen and readily produce silk. It is possible to produce reciprocal crosses with “Line A” to increase the numbers of individuals that are available for the next screening step. Crosses are made each direction (onto the haploid plants and onto “Line A”) to maximize the amount of seed produced. The reciprocal crosses onto “Line A” would produce large amounts of kernels compared to the cross onto the haploid plants. For example, a theoretical reciprocal cross would yield as follows: “Line A”×haploid 500 to 1,000 kernels and haploid×“Line A”=300 Kernels which would generate 800 to 1,300 kernels for advancement.

Next, in the fourth phase, the goal is to maximize percent recurrent parent and the options of the second phase are repeated. In one embodiment, at least one generation of backcrossing to Line A, followed by selection for progeny with maximum percent RP, is conducted. In a preferred aspect, either individual seeds or bulks are sampled using high throughput, non-destructive seed sampling to confirm the presence of each of the transgenic traits and identify seed with maximum percent RP in order to economize plots and expedite time to achievement of product concept.

In another embodiment, the haploid induction process introduced at the second phase is repeated. Induction of more plants can be conducted in a way similar to the second phase, but average RP would be higher. The present invention contemplates that with adequate sample size, it will be possible to identify individuals that contain all four transgenic traits and are greater than 98% RP to advance. In one aspect, these individuals will undergo induction as above. The greater the number of traits involved, the larger number of plants that are used for induction at this step. For example, if the 1000 kernels produced in Option 1 or Option 3 were induced the following would occur: 1,000 kernels×150=150,000 seeds produced; 150,000 seeds×0.05 induction=7500 haploids; 7500 haploids/16=470 with 4 transgenic traits; 470/2=235 with all 4 transgenic traits and recurrent parent greater than 98%. Putative haploid kernels are identified using visual screening, phenotypic screening, and/or genotypic screening using methods known in the art. In a preferred aspect of the present invention, each of the putative haploid kernels is sampled using high throughput, non-destructive seed sampling to determine that each of the transgenic traits of the donor is present and that recurrent parent (RP) is maximized before planting in order to economize plots. Theoretically, 1 of 16 of the putative haploid kernels produced will contain all four traits ( 2250/16=140 putative haploid kernels that have all four transgenic traits) and, on average, at least half of these will be higher than 95% RP (70 putative haploid kernels).

If the fourth phase included induction, in the fifth phase the selected putative haploids are identified using visual screening, phenotypic screening, and/or genotypic screening using methods known in the art. In a preferred aspect of the present invention, each of the putative haploid kernels is sampled using high throughput, non-destructive seed sampling to determine that each of the transgenic traits of the donor is present and that recurrent parent (RP) is maximized before planting in order to economize plots and doubling. Resulting lines are advanced in the breeding pipeline. For example, resulting lines may be used in line and variety development and hybrid development. They may be evaluated for selection of one or more preferred transgenic events based on haplotype effect estimates. One or more resulting lines may be used in transgenic trait integration as a transgenic trait donor. In other aspects, resulting lines may be used in breeding crosses and in testing and advancing a plant through self fertilization. In another aspect, resulting lines segregating for at least one locus are advanced as sister lines. In still other aspects, resulting lines and parts thereof may be used for transformation, for candidates for expression constructs, and for mutagenesis.

### Example 2

### Stacking at Least Two Genetic Factors Using a Haploid Approach and Cytoplasmic Sterility Backcrossing

Notably, the number of transgenic traits and/or genetic factors that are required for a given product concept in this invention will dictate the number of individuals required for screening in order to increase the probability of acquiring target individuals for advancement that comprise the transgenic traits as well as, if relevant, desired percent recurrent parent. There is tremendous value in the hybrid corn market for products with at least two transgenic traits, such as herbicide tolerance and insect resistance. However, traditional backcross methods will result in an exponential increase in resources needed to deliver hybrids with two or more genetic factors, in terms of years to market, plots needed, etc. In the present example, the methods of this invention are detailed, wherein an expedited approach for breeding and transgenic trait integration are provided that leverage cytoplasmic male sterility (CMS).

Cytoplasmic sterility backcrossing is extremely important in the reduction of cost of goods. Traditionally, transgenic trait conversions have been nearly completed before the incorporation of sterility is considered. The present invention provides methods for the parallel integration of CMS and the genetic factors of interest.

In the first generation, the F1 is made by crossing a CMS four trait donor by “Line A”. For purposes of illustration, 500 kernels of this cross are produced. If a correct cytoplasm is chosen, all of the seed produced should be male sterile the ensuing generation. In the second generation, the male sterile F1 is planted in a maternal induction crossing situation using the F1 as female. If the 500 kernels from above are planted in a KHI1 isolation, one would estimate that 75,000 seeds would be produced (500 plants×150 seeds per ear=75,000 seeds) and, of these, approximately 3,500 to 4,000 of these would be putative haploids (75,000 induced seeds×0.05 induction=3,750 putative haploids). Putative haploid kernels are identified using visual screening, phenotypic screening, and/or genotypic screening using methods known in the art. In a preferred aspect of the present invention, each of the putative haploid kernels is sampled using high throughput, non-destructive seed sampling to determine that each of the transgenic traits of the donor is present and that recurrent parent (RP) is maximized before planting in order to economize plots.

In the third generation, the selected putative haploid kernels are planted in a nursery next to “Line A.” The selected haploid kernels are used as female, as they are cytoplasmically male sterile, and are crossed by “Line A”. Haploid plants, which have not been doubled, should be 100% male sterile, but readily produce silk. Assuming correct selection of putative haploids, each of these seeds contains all four transgenic traits and is, at a minimum, 95% recurrent parent. There would be advantage in using “Line A—4 Trait Conversion” as the donor at this stage if available.

It is also possible, if run concurrently, to use pollen from the reciprocal crossing approach as male onto these putative haploid kernels to accelerate the inbreeding and reinforce the four transgenic traits of interest.

The fourth generation is a reiteration of the second generation with expected increased percent RP recovered. The new F1 is planted in a maternal induction crossing situation using the F1 (which is cytoplasmically male sterile) as female. Putative haploid kernels are identified using visual screening, phenotypic screening, and/or genotypic screening using methods known in the art. In a preferred aspect of the present invention, each of the putative haploid kernels is sampled using high throughput, non-destructive seed sampling to determine that each of the transgenic traits of the donor is present and that recurrent parent (RP) is maximized before planting in order to economize plots.

In the fifth generation, putative haploids are sent to a crossing nursery and are planted in close proximity to Line A or, preferably, “Line A—4 Trait Conversion”. The haploid plants are crossed by the “Line A—4 Trait Conversion” which serves as the maintainer. If “Line A—4 Trait Conversion” is undergoing the doubling process concurrently, pollen from the doubled haploids can be used as the donor to these male sterile doubled (or undoubted) cytoplasmic sterile haploid plants. “Line A—4 Trait Conversion” acts as the maintainer to increase the cytoplasmic male sterile version.

In the sixth generation, candidate material with the transgenic traits, CMS, and at least 98% recurrent parent is advanced in the breeding program. For example, resulting lines may be used in line and variety development and hybrid development. They may be evaluated for selection of one or more preferred transgenic events based on haplotype effect estimates. One or more resulting lines may be used in transgenic trait integration as a transgenic trait donor. In other aspects, resulting lines may be used in breeding crosses and in testing and advancing a plant through self fertilization. In another aspect, resulting lines segregating for at least one locus are advanced as sister lines. In still other aspects, resulting lines and parts thereof may be used for transformation, for candidates for expression constructs, and for mutagenesis.

We claim:

1. A method for incorporating at least two genetic factors into at least one plant, the method comprising:
providing crossing a donor plant comprising at least two genetic factors with the at least one plant to obtain a plurality of progeny plants;
crossing at least one of the plurality of progeny plants with a haploid inducer line to produce a plurality of induced progeny comprising haploid progeny;
selecting haploid progeny from the plurality of induced progeny:
screening the selected haploid progeny for the presence of at least one marker for the at least one of the at least two genetic factors and at least one marker for the genome of the at least one plant; and
selecting haploid progeny based on the results of the screening.

2. The method of claim 1, wherein the method further comprises doubling the haploid progeny selected on the basis of the screening results to produce diploid progeny.

3. The method of claim 1, wherein the donor plant and the at least one plant are at least 50% genetically identical.

4. The method of claim 2, wherein the donor plant and the at least one plant are at least 80% genetically identical.

5. The method of claim 3, wherein the donor plant and the at least one plant are at least 90% genetically identical.

6. The method of claim 1, wherein the step of screening the selected haploid progeny comprises screening the haploid progeny for the presence of at least one marker selected from the group consisting of a genetic marker, a haplotype, a nucleic acid sequence, a transcriptional profile, a metabolic profile, a nutrient composition profile, a protein expression profile, and a phenotypic character.

7. The method of claim 1 wherein the step of screening the selected haploid progeny comprises removing a tissue sample from the selected haploid progeny using high throughput, non-destructive seed sampling.

8. The method of claim 2, wherein the step of doubling the selected haploid progeny comprises contacting the selected haploid progeny with a doubling treatment selected from the group consisting of nitrous oxide gas, anti-microtubule herbicides, anti-microtubule agents, colchicine, pronamide, and mitotic inhibitors.

9. The method of claim 8, wherein doubling treatments are applied to one or more parts of the plant selected from the group consisting of cells, tissues, seed, embryos, seedlings, leaves, body, and stem.

10. The method of claim 1, wherein the at least one plant is inbred.

11. The method of claim 1, wherein the at least one plant is segregating at one or more loci.

12. The method of claim 1, wherein the at least one plant comprises at least one genetic factor.

13. The method of claim 1, wherein the method further comprises conducting at least one generation of backcross selection for the at least one plant following any cross.

14. The method of claim 2, wherein the method further comprises using the doubled progeny in one or more germplasm improvement activities selected from the group consisting of line and variety development, hybrid development, transgenic event selection, transgenic trait donor development, making breeding crosses, testing and advancing a plant through self fertilization, using plant or parts thereof for transformation, using plants or parts thereof for candidates for expression constructs, and using plant or parts thereof for mutagenesis.

15. The method of claim 1, wherein the plant is a crop plant selected from the group consisting of maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops.

16. The method of claim 1, wherein at least one of the at least two genetic factors confers a trait selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, sterility, other agronomic traits, traits for industrial uses, and traits for improved consumer appeal.

17. A plant or parts thereof generated by the method of claim 1.

18. A plant or parts thereof generated by the method of claim 2.
